(19) United States
(12) Patent Application Publication
(10) Pub. No.: US 2025/0160801 A1
(43) Pub. Date: May 22, 2025

(54) URINE COLLECTION DEVICE WITH ERGONOMIC FEATURES

(71) Applicants: Garrett Wood, Bow, NH (US); Jeff Kusmierz, Bow, NH (US); Laura Wood, Bow, NH (US); Lili Helmuth, Indianapolis, IN (US)

(72) Inventors: Garrett Wood, Bow, NH (US); Jeff Kusmierz, Bow, NH (US); Laura Wood, Bow, NH (US); Lili Helmuth, Indianapolis, IN (US)

(21) Appl. No.: 18/902,854

(22) Filed: Sep. 30, 2024

Related U.S. Application Data
(60) Provisional application No. 63/541,296, filed on Sep. 29, 2023.

(51) Int. Cl. A61B 10/00

(52) U.S. Cl. CPC A61B 10/007

(57) ABSTRACT

A urine collection device comprising an upper finger accessible space in axial alignment with a lower urine collection cup to provide clean and efficient self-administered urine collection for at home testing.

### BACKGROUND OF THE INVENTION

### Field of the Invention

The present invention relates to fluid collection and testing devices, and more particularly to a urine collection device which enables a subject to self-administer the collection of a urine sample without contacting the sample, and securely contains the same for at-home testing purposes.

### Background of the Art

Collection of urine samples from patients for analysis commonly occurs for a variety of reasons. A common concern of medical practitioners and patients alike, however, is contamination of the urine sample, sanitation and cleanliness.

Many testing devices are sold commercially over the counter for home use by individuals wishing to conduct the tests that require urine collection, such as at home pregnancy tests. The home user shares the same concerns of the medical practitioners with respect to such testing, such as avoiding contamination while maintaining sanitation and cleanliness in order to provide the best testing conditions possible.

Accordingly, solutions to the aforementioned issues in the art are needed and desirable, and in particular, it would desirable to provide a urine collection device that satisfactorily addresses the aforementioned concerns.

### SUMMARY OF THE INVENTION

Apparatus, devices and methods of the invention are directed to solving the problems mentioned above, among others. In particular, the device described herein provides a new and improved urine collector with ergonomic and efficiency features configured to be enable a patient to collect their urine samples in a sanitary and non-contaminating manner.

An object of embodiments of the invention is to provide a device to collect a urine sample with ease and cleanliness, which includes a collection portion or cup, a protruding member having an upper portion and a lower portion. The upper portion includes a radially outer wall and opposing radially inner wall forming a tubular finger hold and guard, such as for the user's thumb. The lower portion is adjacent to the rim of the cup and includes a radially outer wall forming a backsplash for urine to slide down into the cup. As shown in this embodiment, the diameter of the tubular member is less than the diameter of the cup so that the radially inner wall does not interfere with urine collection.

Some embodiments of the invention are directed to a urine collection device comprising: an upper portion comprising a tubular member, the tubular member having a cavity defined by a radially inner side wall and a radially outer side wall, and a bottom wall, the tubular member including an upper tubular member opening providing axial access into the cavity; and a lower portion coupled to the upper portion, the lower portion comprising a container with a cylindrical cavity defined by a circular side wall and a container bottom wall, the cylindrical cavity including an upper cylindrical cavity opening providing axial access into the cylindrical cavity, wherein the axial access provided by the upper tubular member opening and the axial access provided by the upper cylindrical cavity are in the same axial direction, and wherein the radially outer side wall of the tubular member is in radial alignment with a portion of the circular side wall of the container.

In some embodiments of the urine collection device, the tubular member has an elliptical cross section.

In some embodiments of the urine collection device, the tubular member has an oval cross section.

In some embodiments of the urine collection device, the bottom wall of the tubular member is tapered downward and radially outward from the radially inner side wall to the circular side wall of the container.

Some embodiments of the invention are also directed to a urine collection device comprising: an upper portion comprising a tubular member, the tubular member having a cavity defined by a radially inner side wall and a radially outer side wall, and a bottom wall, the tubular member including an upper tubular member opening providing axial access into the cavity; and a lower portion coupled to the upper portion, the lower portion comprising a container with a cylindrical cavity defined by a circular side wall and a container bottom wall, the cylindrical cavity including an upper cylindrical cavity opening providing axial access into the cylindrical cavity, wherein the axial access provided by the upper tubular member opening and the axial access provided by the upper cylindrical cavity are in the same axial direction, wherein the radially outer side wall of the tubular member is in radial alignment with a portion of the circular side wall of the container, and wherein the bottom wall of the tubular member is tapered downward and radially outward from the radially inner side wall to the circular side wall of the container.

Other embodiments, features and advantages of the invention will be readily appreciated and apparent from the following detailed description when considered in connection with the accompanying drawings.

### BRIEF DESCRIPTION OF DRAWINGS

While the disclosure concludes with claims particularly pointing out and distinctly claiming specific embodiments, various features and advantages of embodiments within the scope of this disclosure may be more readily ascertained from the following description when read in conjunction with the accompanying drawings, in which:

FIG. 1 is a perspective side view of a urine collection device which has been constructed in accordance with the principles of the invention;

FIG. 2 is a perspective top view of the urine collection device of FIG. 1; and

FIG. 3 is a top view of the urine collection device of FIG. 1.

### DETAILED DESCRIPTION OF SOME EMBODIMENTS OF THE INVENTION

The following detailed description is directed to certain specific embodiments of the invention. However, the invention can be embodied in a multitude of different ways as defined and covered by the claims and their equivalents. In this description, reference is made to the drawings wherein like parts are designated with like numerals throughout.

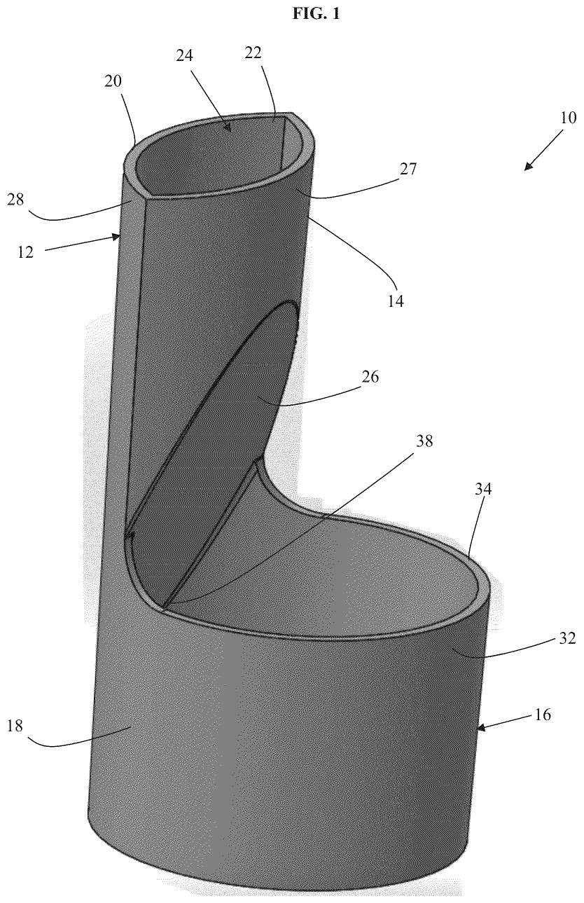

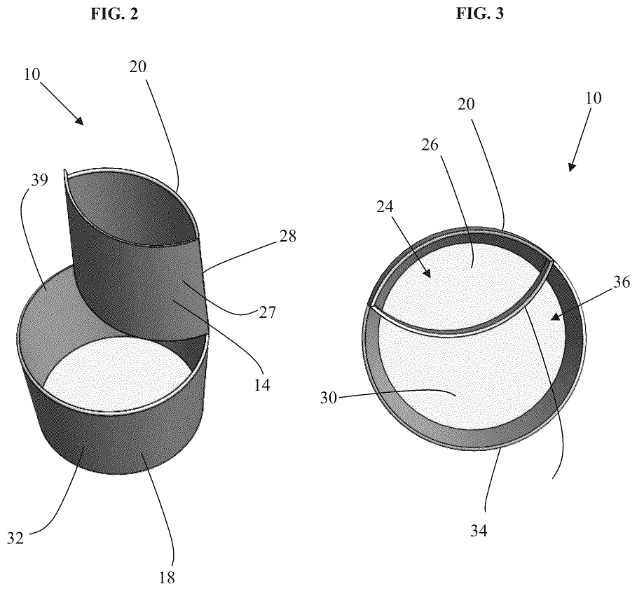

FIG. 1-3 depict a urine sample collection and testing device constructed according to the invention which is generally designated as reference numeral 10. Device 10 includes an upper body portion 12 which comprises a tubular member 14 coupled to a lower body portion 16 which comprises a cup or container 18.

Tubular member 14 has a rim 20 defining an open upper side or opening 22 into a cavity 24 within tubular member 14. Cavity 24 is closed at a bottom 26 of tubular member 12. In this embodiment, upper opening 22 and rim 20 of tubular member 14 have a generally oval or elliptical shaped cross-sectional profile formed by an arcuate radially inner side wall 27 and an arcuate radially outer side wall 28 which also at least partially define cavity 24.

In this embodiment, container 18 is generally cylindrical shaped and includes a bottom 30, circular side wall 32 and rim 34 forming a fluid receiving space or reservoir 36 therein having access thereto through an open upper side 39.

Tubular member 14 and container 18 are positioned axially with the cross-sectional profile of tubular member 14 being nested eccentrically within the circular cross-sectional profile of container 18. The curvature of radially outer side wall 28 of tubular member 14 is configured and dimensioned whereby radially outer side wall 28 of tubular member 14 is coupled to rim 34 in matching radial alignment or parallel with the arc formed by a portion of side wall 32. Cavity 24 and reservoir 36 provide access through opening 22 and opening 39, respectively, which are in axial alignment and provide access thereto from the same axial direction.

In this embodiment, bottom 26 which closes tubular member 12 is sloped or tapered downward and radially outward from inner side wall 27 to be coupled with either radially outer side wall 28, or in this embodiment, an intermediate portion 38 of the inner surface of side wall 32, wherein the intermediate portion 38 is at an axial position between bottom 30 and rim 34 of container 18.

It is envisioned that the downward and radially outward tapered configuration of bottom 26 and other aspects of device 10, such as the oval or elliptical cross-section of tubular member 14, advantageously provide, among other things, sufficient space for holding device 10, such as with the user's thumb, while also sealing and separating cavity 24 from reservoir 36, and therefore avoiding potential contact between the user holding device 10 and urine being collected in reservoir 36 without interfering with or changing the collection volume of reservoir 36.

Rim 20, upper opening 22 and cavity 24 are configured and dimensioned to receive a finger of the user therein, such as the user's thumb. It is envisioned that a user may hold device 10 by inserting one finger within cavity 24, such as the user's thumb, and another finger, such as the pointer on the outer surface of radially outer wall 28 of tubular member 14.

The device of the invention is thus able to keep the user's fingers clean while collecting a urine sample in reservoir 36. For example, the user's thumb may hold device 10 while inserted in the cavity 24 which is closed off to urine entering reservoir 36 by bottom 26. Thus, in practice, a woman may put upper opening 22 of tubular member 14 closer than in prior art devices, such as 2 inches, above her urethra to use device 10 and allow urine to be collected into reservoir 36 of container 18 without spilling or coming into contact with her fingers and possibly being contaminated. It should be readily apparent that an ovulation strip or pregnancy test strip may then be dipped into reservoir 36 for testing.

Device 10 may be constructed of any suitable materials, such as a plastics, or combinations thereof, and may be disposable and biodegradable.

This detailed description of embodiments of the invention are not intended to be exhaustive or to limit the invention to the precise form disclosed above.

It should be understood that, the described features, structures, or characteristics of the invention may be combined in any suitable manner in one or more embodiments. The description of the invention includes numerous specific details to provide a thorough understanding of embodiments of the invention. One skilled in the relevant art will recognize, however, that the invention can be practiced without one or more of the specific details, or with other methods, components, materials, and so forth.

It should be further understood that features and components in the figures are not necessarily to scale, and certain features and components are included, whether displayed or not, and made part of this disclosure, but not shown or specifically discussed herein with reference to the drawings, emphasis instead being placed upon illustrating the principles of the invention and depicted embodiments of the invention. In other instances, well-known structures, materials, or operations are not shown or described in detail to avoid obscuring aspects of the invention.

Unless otherwise apparent or stated, directional and relative references, such as “upper,” “lower,”, “upward,” “downward,” “inward”, “front,” “rear,” “frontward,” “rearward,” “distal,” “proximal,” “top,” “bottom” and the like are non-limiting, used for the sake of convenience and ease in explaining some embodiments of the invention, and at most is intended to be relative to the orientation of a particular embodiment of the invention as shown in the figures.

Descriptions made with reference to “embodiment”, “embodiments”, “some embodiments”, “an embodiment”, “various embodiments” or the like mean that the description is applicable to at least one embodiment of the invention but not necessarily all embodiments. The terms “comprising,” “including,” “having,” and the like, as used with respect to one or more embodiments, are synonymous.

As an aid to understanding, various actions, operations or steps may sometimes be described herein in sequence. However, the order of description or written presentation herein is not to be construed to mean or imply that such must necessarily occur in a corresponding order or sequence unless otherwise expressly and clearly stated or logically essential.

While exemplary apparatus, systems and methods of the invention have been described herein, it should also be understood that the foregoing is only illustrative of a few particular embodiments with exemplary and/or preferred features, as well as principles of the invention, and that various modifications can be made by those skilled in the art without departing from the scope and spirit of the invention. Therefore, the described embodiments should not be considered as limiting of the scope of the invention in any way. Accordingly, the invention embraces alternatives, modifications and variations which fall within the spirit and scope of the invention as set forth herein and in the claims, and any equivalents thereto.

1. A urine collection device comprising:
an upper portion comprising a tubular member, the tubular member having a cavity defined by a radially inner side wall and a radially outer side wall, and a bottom wall, the tubular member including an upper tubular member opening providing axial access into the cavity; and
a lower portion coupled to the upper portion, the lower portion comprising a container with a cylindrical cavity defined by a circular side wall and a container bottom wall, the cylindrical cavity including an upper cylindrical cavity opening providing axial access into the cylindrical cavity, wherein the axial access provided by the upper tubular member opening and the axial access provided by the upper cylindrical cavity are in the same axial direction, and wherein the radially outer side wall of the tubular member is in radial alignment with a portion of the circular side wall of the container.

2. The urine collection device of claim 1, wherein the tubular member has an elliptical cross section.

3. The urine collection device of claim 1, wherein the tubular member has an oval cross section.

4. The urine collection device of claim 1, wherein the bottom wall of the tubular member is tapered downward and radially outward from the radially inner side wall to the circular side wall of the container.

5. A urine collection device comprising:
an upper portion comprising a tubular member, the tubular member having a cavity defined by a radially inner side wall and a radially outer side wall, and a bottom wall, the tubular member including an upper tubular member opening providing axial access into the cavity; and
a lower portion coupled to the upper portion, the lower portion comprising a container with a cylindrical cavity defined by a circular side wall and a container bottom wall, the cylindrical cavity including an upper cylindrical cavity opening providing axial access into the cylindrical cavity, wherein the axial access provided by the upper tubular member opening and the axial access provided by the upper cylindrical cavity are in the same axial direction, wherein the radially outer side wall of the tubular member is in radial alignment with a portion of the circular side wall of the container, and wherein the bottom wall of the tubular member is tapered downward and radially outward from the radially inner side wall to the circular side wall of the container.

6. The urine collection device of claim 1, wherein the tubular member has an elliptical cross section.

7. The urine collection device of claim 1, wherein the tubular member has an oval cross section.
